(19) United States
(12) Patent Application Publication
(10) Pub. No.: US 2009/0164020 A1
(43) Pub. Date: Jun. 25, 2009

(54) Device for Securing an Implant to Tissue

(75) Inventors: Brian Janowski, Marquette, MI (US); John Kovarik, Negaunee, MI (US); Wade DePas, Ishpeming, MI (US); Jeffrey Trudeau, Marquette, MI (US)

Correspondence Address: FITCH EVEN TABIN AND FLANNERY, 120 SOUTH LASALLE STREET, SUITE 1600, CHICAGO, IL, 60603-3406, US

(73) Assignee: PIONEER SURGICAL TECHNOLOGY, INC., Marquette, MI (US)

(21) Appl. No.: 12/324,292

(22) Filed: Nov. 26, 2008

Related U.S. Application Data
(60) Provisional application No. 60/990,809, filed on Nov. 28, 2007.

(51) Int. Cl. A61F 2/44; A61B 17/70

(52) U.S. Cl. 623/17.16; 606/247

(57) ABSTRACT

An implant device is provided for implantation within an intervertebral space between adjacent vertebrae comprising an implant body, a rotatable portion and a piercing portion configured to pierce the adjacent vertebra.

### CROSS REFERENCE TO RELATED APPLICATIONS

This application claims the benefit of the filing date of U.S. Provisional Application 60/990,809, which is hereby incorporated in its entirety herein.

### FIELD OF THE INVENTION

The invention relates to implant devices for implantation within an intervertebral space and fixation to the adjacent vertebrae.

### BACKGROUND OF THE INVENTION

The spine is the central support column for the human body. It includes a series of vertebrae and intervertebral discs between adjacent vertebrae. The vertebrae are formed of hard bone while the intervertebral discs comprise a comparatively soft annulus and nucleus. The intervertebral discs help to absorb pressure, distribute stress, and keep adjacent vertebrae from grinding against each other.

A variety of spinal conditions including, for example, trauma, deformity, disease, or other degenerative conditions, may result in a person experiencing pain or limited physical mobility. This pain and reduced mobility is often attributed to the rupture or degeneration of the intervertebral discs resulting in compression of spinal nerve roots.

One manner of treating these conditions is through immobilization and fusion of the injured portion of the spine. In spinal fusion surgery, two or more adjacent vertebrae are initially immobilized relative to each other and, over time, become fused in a desired spatial relationship. Often, these procedures require correcting the spacing between adjacent vertebrae by implanting an intervertebral implant.

One problem with existing intervertebral implants is that, once inserted, the implants are explanted from between adjacent vertebrae. To promote immobilization and fusion of adjacent vertebrae, the intervertebral implant should be designed to provide a substantially flush interface with the endplates of the adjacent vertebrae. However, studies have shown that the vertebral endplates of the lumbar spine have varying degrees of concavity. More specifically, the superior endplates show a tendency to be less concave than the inferior endplates. Accordingly, there is a need for implants that resist explantation from between the adjacent vertebrae and provide for flush engagement with the inferior and superior endplates.

The present invention may be used to fulfill these, as well as other needs and objectives, as will be apparent from the following description of embodiments of the present invention.

### SUMMARY OF THE INVENTION

Thus, in accordance with one aspect of the invention, an implant device is provided for implantation between adjacent vertebrae. The implant device comprises an implant body, a plurality of gripping portions, a rotatable portion and a piercing portion. The implant body includes a leading edge and a trailing edge. The gripping portions extend from the implant body and are configured to engage at least one of the adjacent vertebrae. The rotatable portion of the implant body extends from the leading edge to the trailing edge and defines an axis. The rotatable portion is further configured to be rotatable about the axis when the implant device is positioned between adjacent vertebrae. The piercing portion of the implant device extends from the rotatable portion and is configured to rotate about the axis and rotatably pierce an adjacent vertebra.

According yet another aspect of the invention, an implant device is provided for implantation within an intervertebral device between adjacent vertebrae, which comprises an implant body, a plurality of gripping portions, and a piercing portion. The implant body includes a leading edge and a trailing edge and defines a longitudinal axis therebetween. The gripping portions extend from the implant body and are configured to grip at least one of the adjacent vertebra. The piercing portion is integral with the implant body and extends generally normal to the longitudinal axis. Further, the implant body is configured to rotate between adjacent vertebrae so that the piercing portion rotatably pierces one of the adjacent vertebrae.

### BRIEF DESCRIPTION OF THE DRAWINGS

FIG. 1 is a perspective view of an implant device in accordance with one aspect of the invention;

FIG. 2 is an end view of the trailing edge of the implant device of FIG. 1;

FIG. 3 is a perspective view of the implant device of FIG. 1 with the piercing portions rotated within the central cavity;

FIG. 4 is an end view of the trailing edge of the implant device of FIG. 1;

FIG. 5 is an end view of the leading edge of the implant device of FIG. 1;

FIG. 6 is an end view of the leading edge of the implant device of FIG. 1 with one of the securing wall portions removed;

FIG. 7 is a side view of the implant device of FIG. 1;

FIG. 8 is a top plan view of the implant device of FIG. 1;

FIG. 9 is a perspective view of an implant device in accordance with another aspect of the invention;

FIG. 10 is an end view of the trailing edge of the implant device of FIG. 9;

FIG. 11 is a perspective view of the implant device of FIG. 9 with the piercing portions rotated within the central cavity;

FIG. 12 is an end view of the trailing edge of the implant device of FIG. 9;

FIG. 13 is an end view of the leading edge of the implant device of FIG. 9;

FIG. 14 is an end view of the leading edge of the implant device of FIG. 9 with the securing wall portion removed;

FIG. 15 is a side view of the implant device of FIG. 9;

FIG. 16 is a top plan view of the implant device of FIG. 9;

FIG. 17 is a perspective view of an implant device in accordance with another aspect of the invention;

FIG. 18 is an end view of the trailing edge of the implant device of FIG. 17;

FIG. 19 is a perspective view of the implant device of FIG. 17 with the piercing portions rotated within the central cavity;

FIG. 20 is an end view of the leading edge of the implant device of FIG. 17;

FIG. 21 is an exploded perspective view of the implant device of FIG. 17;

FIG. 22 is a perspective view of an implant device in accordance with another aspect of the invention;

FIG. 23 is a perspective view of an implant device in accordance with another aspect of the invention;

FIG. 24 is a perspective view of an implant device in accordance with another aspect of the invention;

FIG. 25 is a side view of the implant device of FIG. 24;

FIG. 26 is an end view of the leading edge of the implant device of FIG. 24;

FIG. 27 is an end view of the trailing edge of the implant device of FIG. 24;

FIG. 28 is a top plan view of the implant device of FIG. 24;

FIG. 29 is a perspective view of an implant device in accordance with another aspect of the invention;

FIG. 30 is a side view of the implant device of FIG. 29;

FIG. 31 is a top plan view of the implant device of FIG. 29;

FIG. 32 is a perspective view of a spine; and

FIG. 33 is a perspective view of the insertion tool.

### DETAILED DESCRIPTION OF THE PREFERRED EMBODIMENTS

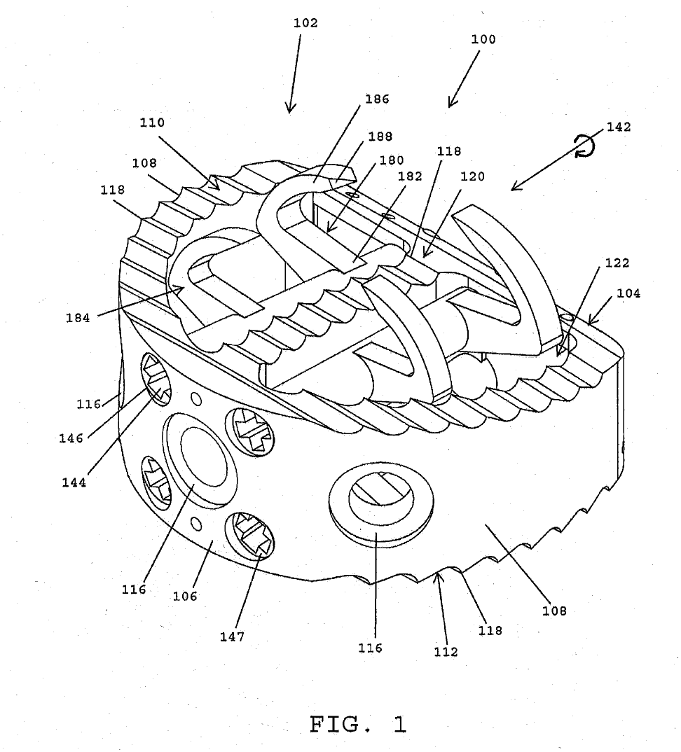

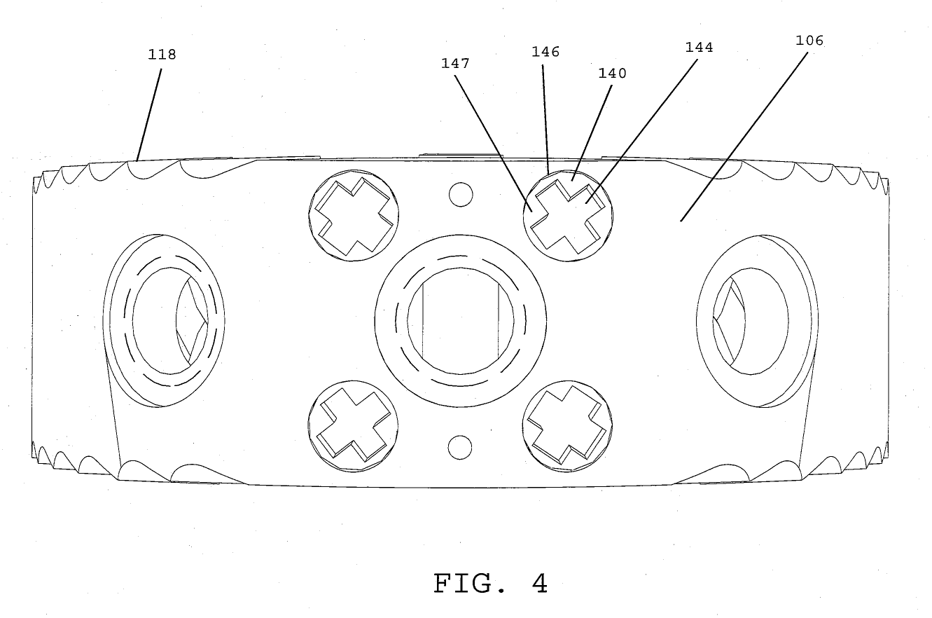

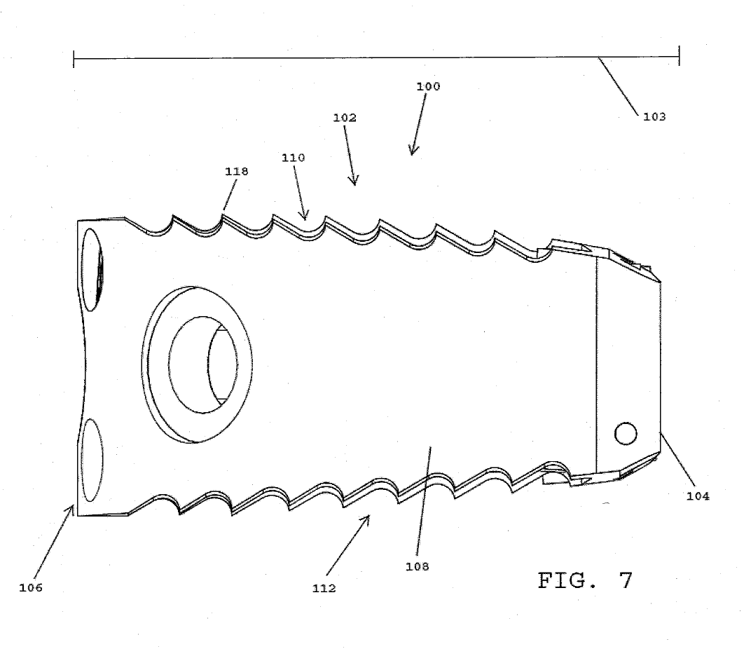

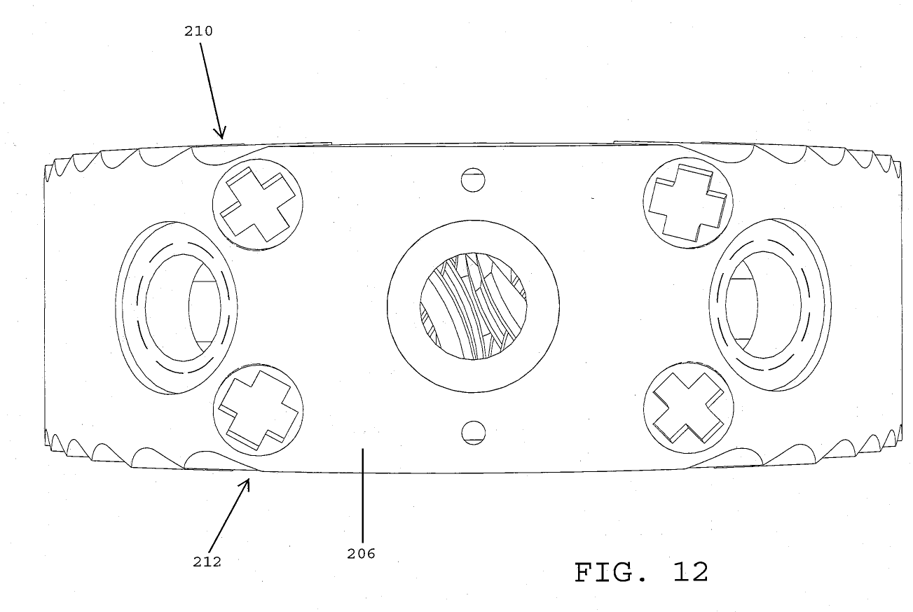

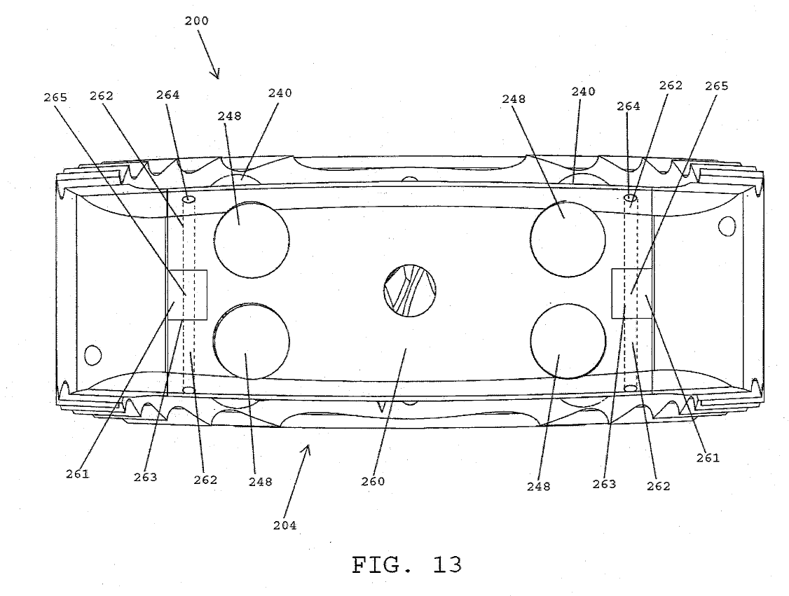

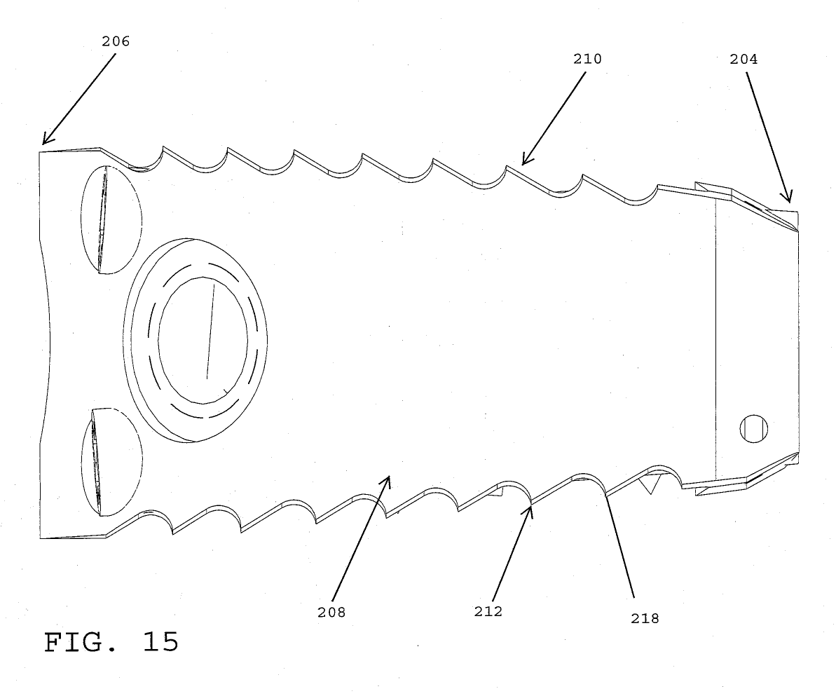

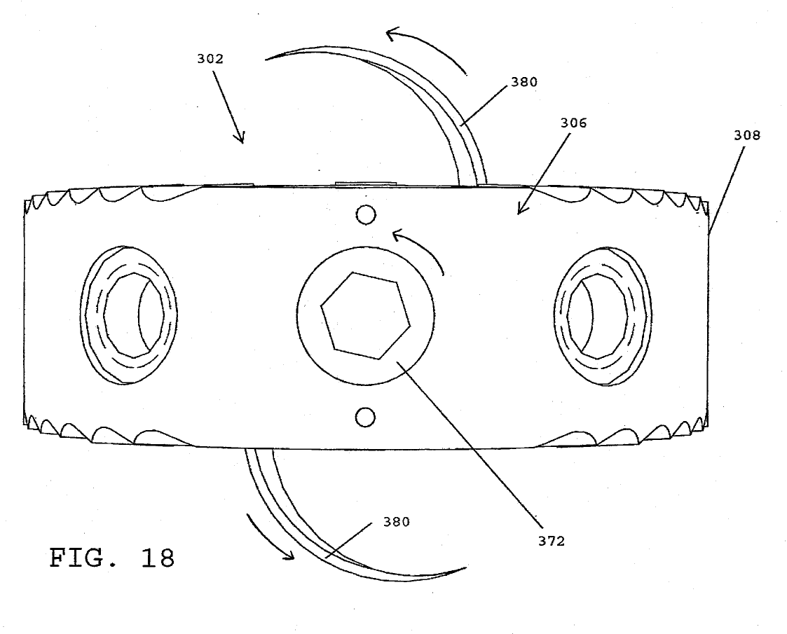

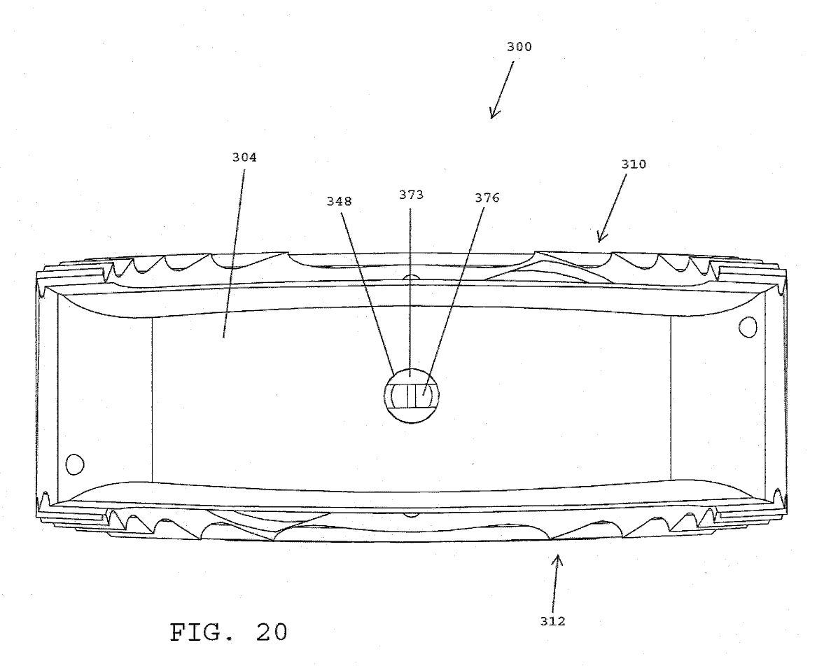

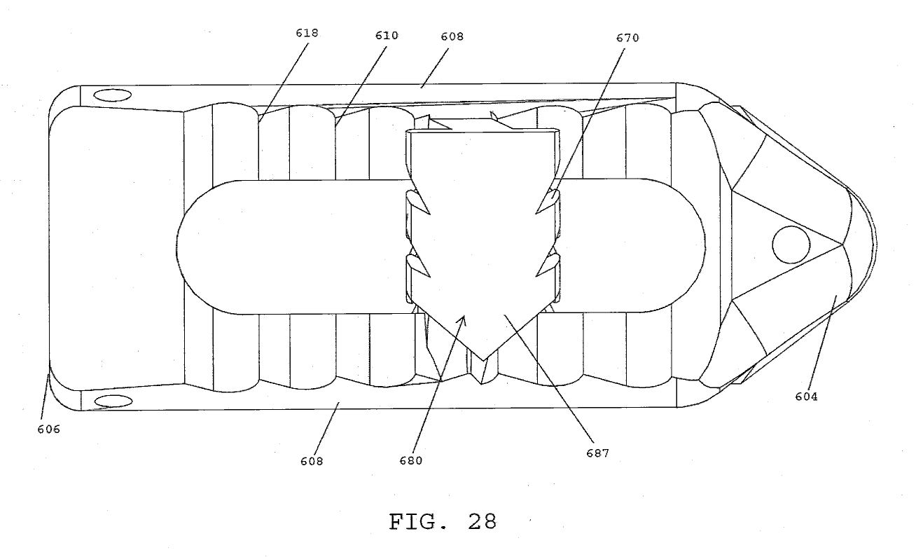

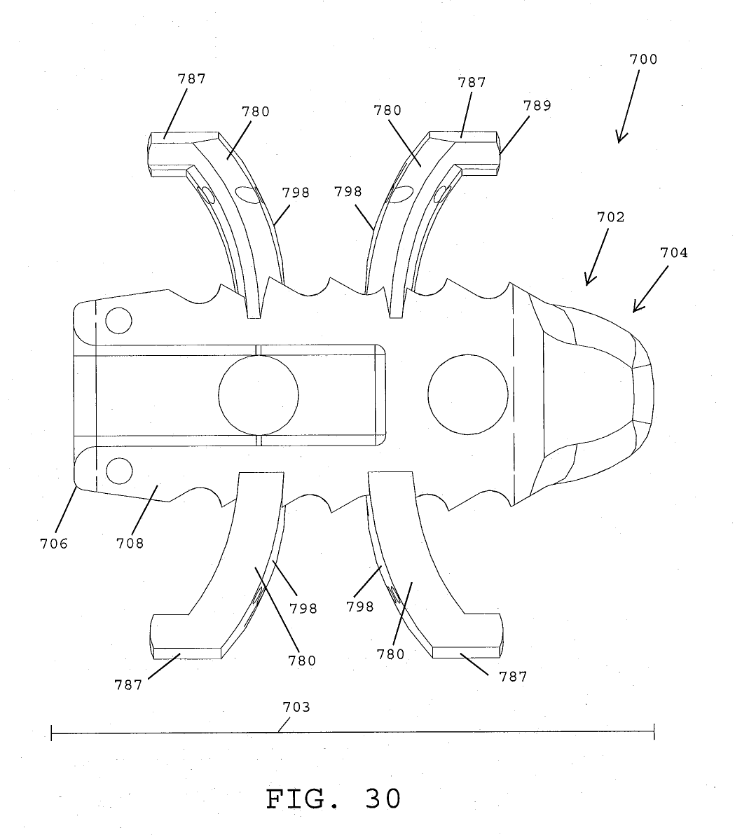

With reference to FIGS. 1-31, an implant device is shown configured in accordance with various aspects of the invention for being implanted within the spine 6 between adjacent vertebral bodies 10 and secured to at least one of those bodies 10. Further contemplated embodiments include artificial discs, annulus plugs, and other implants, such as those described in U.S. Patent Application Publication No. 2006/0129238 to Paltzer, U.S. Patent Application Publication No. 2007/0282441 to Stream et al., and U.S. Patent Application Publication No. 2008/0103598 to Trudeau et al., which are hereby incorporated in their entirety herein.

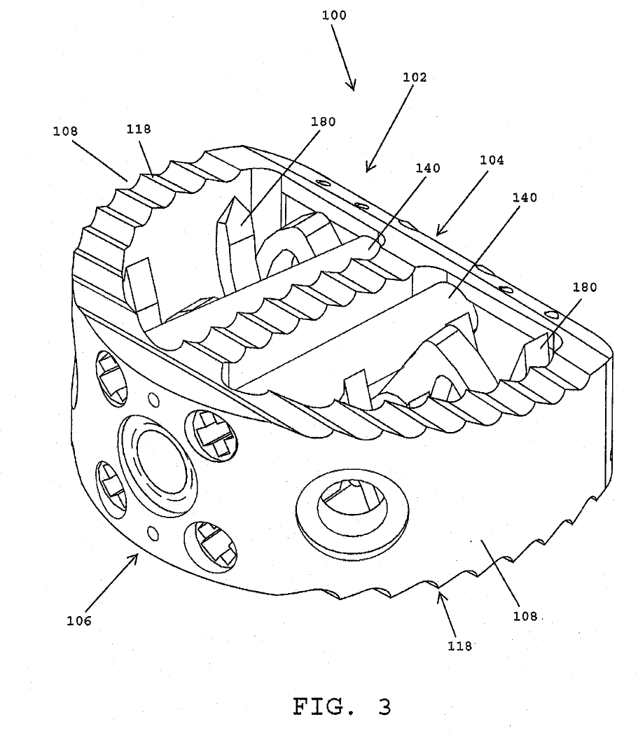

With reference to FIGS. 1-8, the implant device 100 is shown in accordance with one aspect of the invention. The implant device 100 includes an implant body 102, a rotatable portion 140 and a piercing portion 180 extending from the rotatable portion 140. The rotatable portion 140 and piercing portion 180 can be arranged in a compact orientation, as shown in FIG. 3, an extended orientation, as shown in FIG. 1, or an intermediate orientation. The rotatable portion 140 and piercing portion 180 are configured to provide adequate structural strength to the implant device 100 so that adequate torque can be applied so the piercing portion 180 can penetrate the adjacent vertebral body 10.

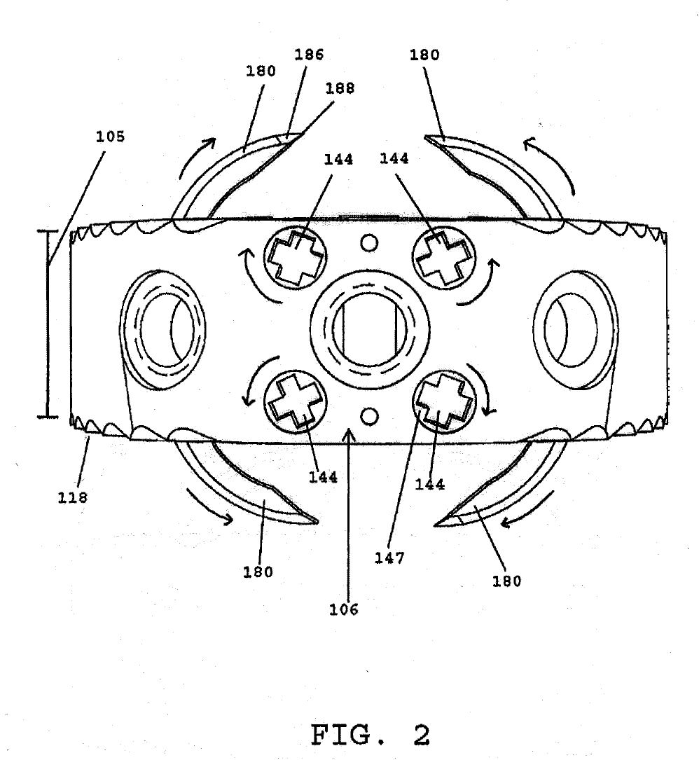

The rotatable portion 140 extends from the leading edge 104 of the implant body 102 to the trailing edge 106 of the implant body 102 and defines a longitudinal axis 142. In one embodiment, the rotatable portion 140 extends parallel to one of the upper and lower surfaces 110, 112 of the implant body 102. In an alternative embodiment, the rotatable portion 140 extends across the implant body 102 in a direction which is not parallel to either the upper or lower surfaces 110, 112. As shown in FIGS. 1-8, the rotatable portion 140 preferably extends through a throughbore 146 in the trailing edge 106 and a throughbore 148 in the leading edge 104. The throughbores 146, 148 are preferably located generally centrally between the lateral edges 108 of the implant body 102, as shown in FIG. 2. Further, the throughbores 146, 148 can be located along the height 105 of the implant body 102. In one embodiment, as shown in FIGS. 1-4, the throughbores 146, 148 are adjacent either the upper surface 110 or lower surface 112 of the implant body 102.

The rotatable portion 140 and the throughbores 146, 148 are configured to permit rotation of the rotatable portion 140 within the throughbores 146, 148. Preferably, the throughbores 146, 148 include a smooth annular surface, as shown in FIGS. 2, 3, and the rotatable portion 140 includes corresponding annular surfaces at either end. In the illustrated embodiment, the rotatable portion 140 includes an annular surface along the entire length of the rotatable portion 140. Other configurations, such as the use of a bearing or bushing between the rotatable portion 140 and throughbores 146, 148, are contemplated to ease and enable rotation of the rotatable portion 140.

Preferably, the trailing end 147 of the rotatable portion 140 includes a tool engagement portion 144. The tool engagement portion 144 is configured to be engaged by a tool apparatus 1000 to rotate the rotatable portion 140 and the piercing portion 180 extending therefrom about the longitudinal axis 142. The rotatable portion 140 and tool engagement portion 144 are configured to deliver sufficient torque to the piercing portion 180 to permit the piercing portion 180 to rotatably penetrate the adjacent vertebral body 10. In one embodiment, as shown in FIGS. 1-4, the tool engagement portion 144 includes an X-shaped aperture in the trailing end 147 of the rotatable portion 140.

The rotatable portion 140 and implant body 102 are further configured to permit the rotatable portion 140 to be positioned within the implant body 102. Various configurations include, for example, a collapsible rotatable portion 140, an expandable implant body 102, and one or both of the rotatable portion 140 and implant body 102 comprising more than one member thereby allowing for disassembly prior to positioning of the rotatable portion 140 within the implant body 102 and reassembly upon positioning of the rotatable portion 140 in the desired location.

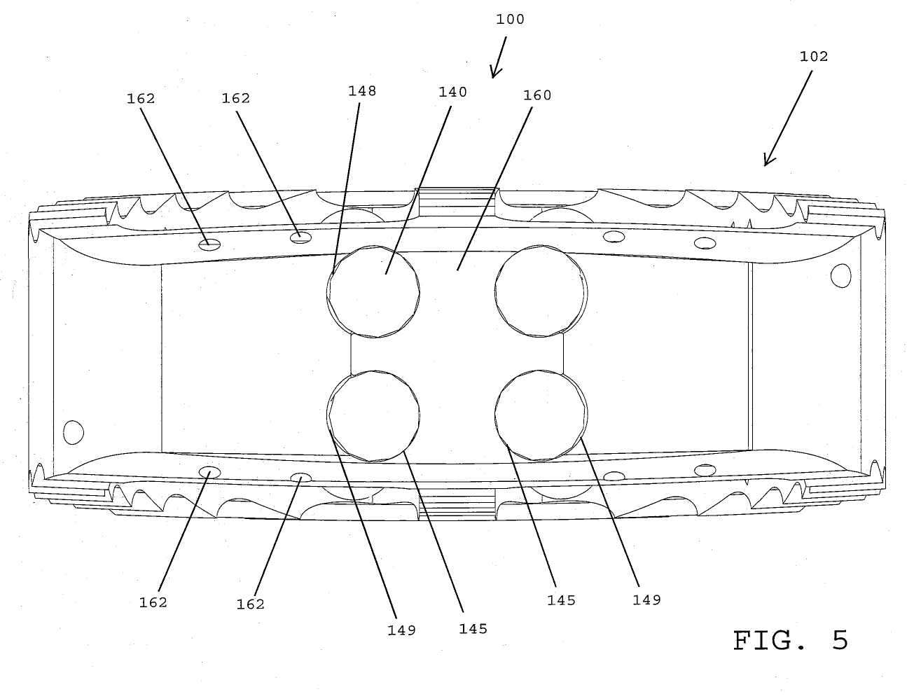

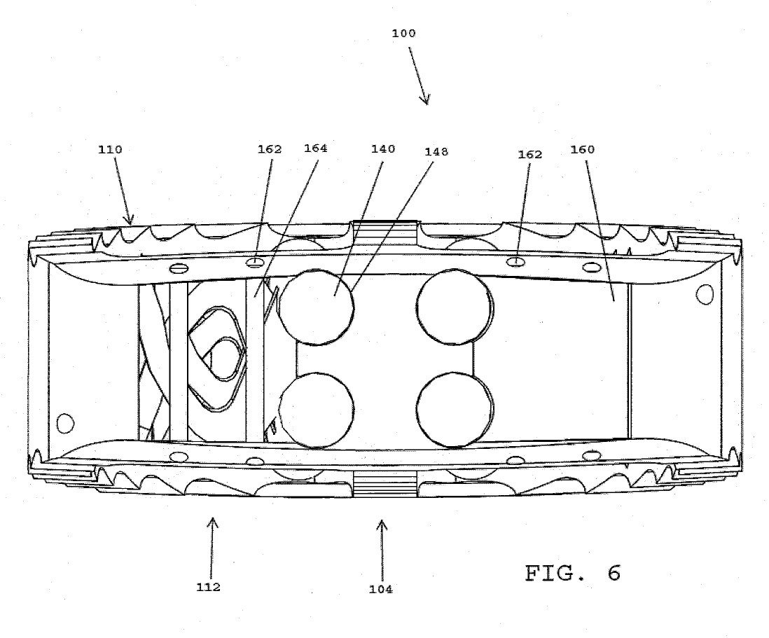

In one embodiment, as shown in FIGS. 5 and 6, the leading edge 104 of the implant body includes a removable securing wall portion 160. The leading edge 104 and removable securing wall portion 160 define the throughbore 148. Preferably, the throughbore 148 is defined by a penannular portion 145 configured to accept the rotatable portion 140 and a rounded portion 149 configured to secure the rotatable portion 140 in the penannular portion 145. In a preferred embodiment, the leading edge 104 includes the penannular portion 145 to permit the rotatable portion 140 to be positioned within both the throughbore 446 and the penannular portion 145 of throughbore 148 before the securing wall 160 is secured to the leading edge 104. The removable securing wall portion 160 is configured to be secured onto the leading edge 104 by any known means. Preferably, as shown in FIGS. 5, 6, the leading edge 104 includes securing throughbores 162, and the removable securing wall portion 160 includes corresponding securing throughbores 163, the securing throughbores 162, 163 configured to receive a securing member 164, such as a pin, therein, to secure the removable securing wall portion 160 to the leading edge 104.

The piercing portion 180 includes a proximal portion 182, which extends from the rotatable portion 140, and a distal end portion 186. In one embodiment, the proximal portion 182 is integral with the rotatable portion 140. In an alternative embodiment, the proximal portion 182 is secured to the rotatable portion 140 by any known means, such as, for example, a screw, an interlocking mechanism of the proximal portion 182 and the rotatable portion 140, or by an adhesive. Preferably, the distal end portion 186 includes a tapered end portion 188 to ease the penetration of the distal end portion 186 of the piercing portion 180 into the vertebral body 10.

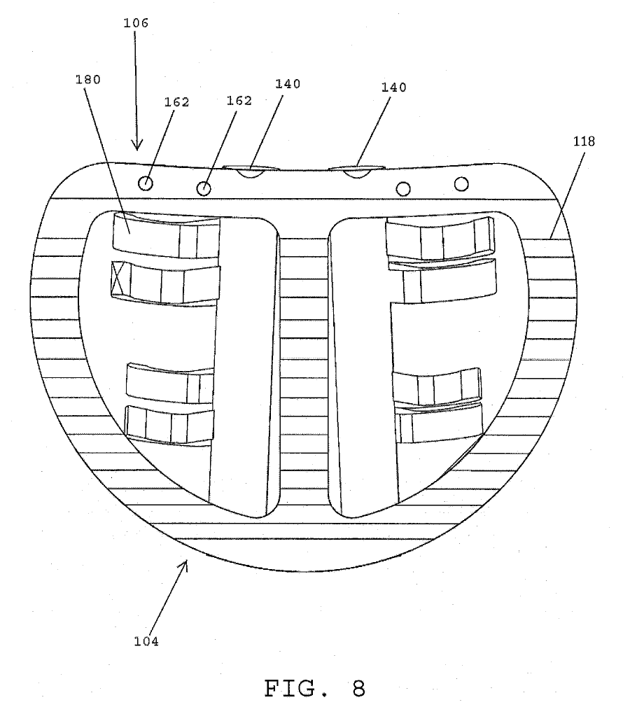

In the insertion orientation, the piercing portion 180 is located within a central cavity 122 of the implant body 102, which extends from the upper surface 110 of the implant body 102 to the lower surface 112 of the implant body 102, and from the leading edge 104 to the trailing edge 106. In one embodiment, the central cavity 122 extends from one lateral edge 108 to the other lateral edge 108. In a preferred embodiment, as shown in FIG. 8, the central cavity 122 extends from one lateral edge 108 to a central support portion 120. The central support portion 120 extends from the leading edge 104 to the trailing edge 106 and is generally intermediate the lateral edges 108. Preferably, the central support portion 120 extends from the upper surface 110 of the implant body 102 to the lower surface 112 of the implant body 102 and is configured to engage and support the adjacent vertebral bodies 10.

In the securing orientation, the piercing portion 180 extends away from one of the upper and lower surfaces 110, 112 of the implant body 102. The piercing portion 180 is configured to extend above the upper surface 110 or lower surface 112 a distance sufficient to secure the implant body 102 to the vertebral body 10 without compromising the integrity of the vertebral body 10.

As the rotating portion 140 and piercing portion 180 are rotated between adjacent vertebral bodies 10, the piercing portion 180 extends, for example, above the upper surface 110, out of the central cavity 122 toward the adjacent vertebral body 10 and, as it does so, penetrates the vertebral body 10. As the piercing portion 180 rotatably penetrates the vertebral body 10, the implant body 102 is urged toward the vertebral body 10 until, preferably, the upper surface 110 firmly engages the vertebral body 10.

In one embodiment, the implant device 100 includes one piercing portion 180 extending from one rotatable portion 140. In an alternative embodiment, the implant device 100 includes at least two piercing portions 180 extending from a rotatable portion 140, the piercing portions 180 preferably extending in parallel from the rotatable portion 140. In a further preferable embodiment, and as shown in FIGS. 1-4, the implant device 100 includes at least two rotatable portions 140, with one or more piercing portions extending from each rotatable portion 140. In another embodiment, the implant device 100 includes two rotatable portions 140 with corresponding piercing portions 180 configured to extend beyond one of the upper and lower surfaces 110, 112 of the implant body 102. In a preferable embodiment, the implant device 100 includes at least two rotatable portions 140 with corresponding piercing portions 180, at least one rotatable portion 140 with corresponding piercing portions 180 configured to extend from each of the upper and lower surfaces 110, 112 of the implant body 102.

As shown in FIGS. 1, 3, 8, in a preferred embodiment the rotatable portions 140 and piercing portions 180 are configured to not interfere with one another in the insertion orientation or the securing orientation. In particular, the piercing portions 140 can be positioned within the central cavity 122 with the piercing portions 180 staggered along the longitudinal axis 142 of the rotatable portions 140 so that all of the piercing portions 180 can be disposed within the central cavity 122 in the insertion orientation, preferably with the piercing portions 180 positioned generally between the upper and lower surfaces 110, 112 of the implant body 102 to assist in insertion of the implant body 102 between adjacent vertebral bodies 10.

The configuration of the piercing portion 180 is dependent on multiple variables including, for example, the width, depth and height of the central cavity 122, the location of the rotatable members 140 and corresponding throughbores 146, 148 within the central cavity 122, and the number of rotatable members 140 and piercing portions 180 in the implant device 100. Additional variables include the shape, length, width and depth of the piercing portions 180 and the direction in which the piercing portions 180 extend. In particular, repositioning the rotatable members 140 and throughbores 146, 148 along the height and width of the central cavity 122 can be used to accommodate varying piercing portion 180 configurations including, for example, differences in shape, length, depth, width, and direction in which the piercing portions 180 extend.

Additionally, the particular shape of the piercing portion 180 can depend on factors such as the density of the bone to be penetrated, the degree of compression required between the device and the bone, the static and dynamic loading on the implant and bone, as well as the strength of the materials used.

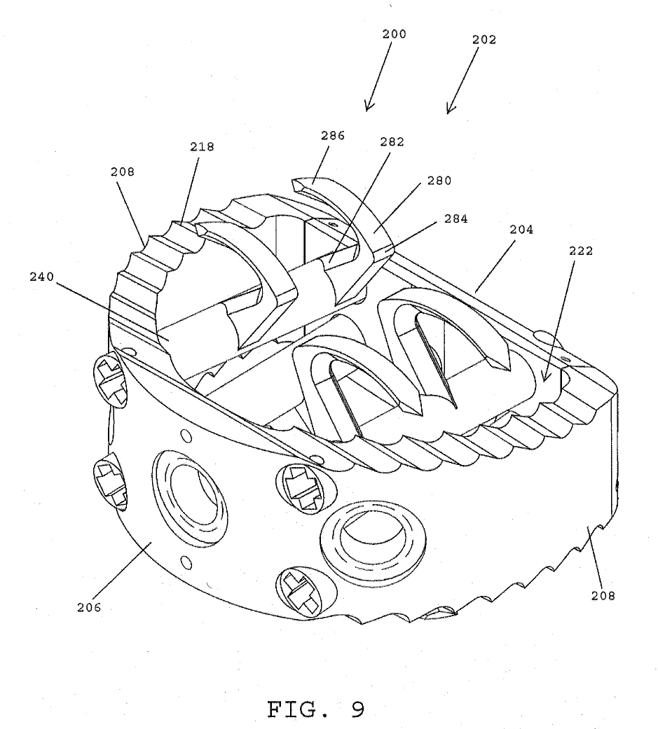

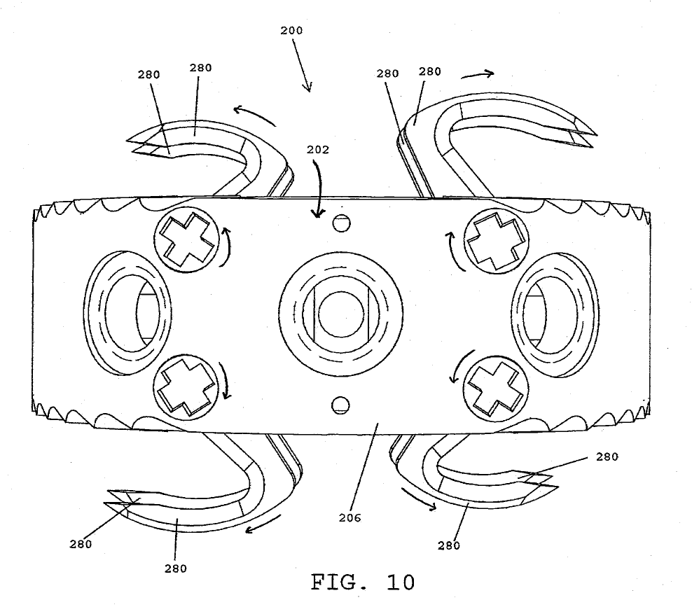

As shown in FIGS. 1, 2, the piercing portions 180 extend generally away from the nearest lateral edge 108 and toward the center of the implant body 102. Alternative embodiments include, for example, piercing portions 180 extending away from the center of the implant body 102 (as shown in FIGS. 9, 10), all the piercing portions 180 extending in the same direction, or the piercing portions 180 extending in a plurality of directions. Preferably, the piercing portions 180 extend in at least two different directions to provide additional stability in securing the implant device 100 to the adjacent vertebrae. In particular, having piercing portions 180 extending in at least two different directions allows the implant body 102 to be secured to the adjacent vertebral body 10 without urging the vertebral body 10 in one direction, thereby avoiding potential damage to the spine and allowing the implant device 100 to be secured in the desired location.

In a preferred embodiment, as shown in FIGS. 1-9, the rotatable portions 140 are positioned adjacent the upper and lower surfaces 110, 112 and toward the lateral edges 108 of the implant body. By positioning the rotatable portions 140 away from the center of the implant body 102, the implant device 100 engages the vertebral bodies at four distinct, spaced locations, providing for a more secure engagement which does not require additional securing methods, such as a pedicle screw or lumbar plate, thereby simplifying the process of securing the vertebral bodies 10 with an implant device 100.

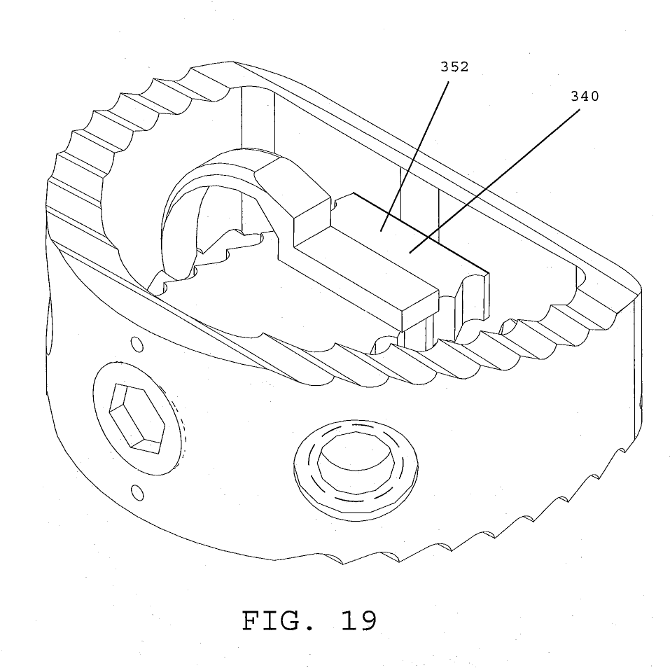

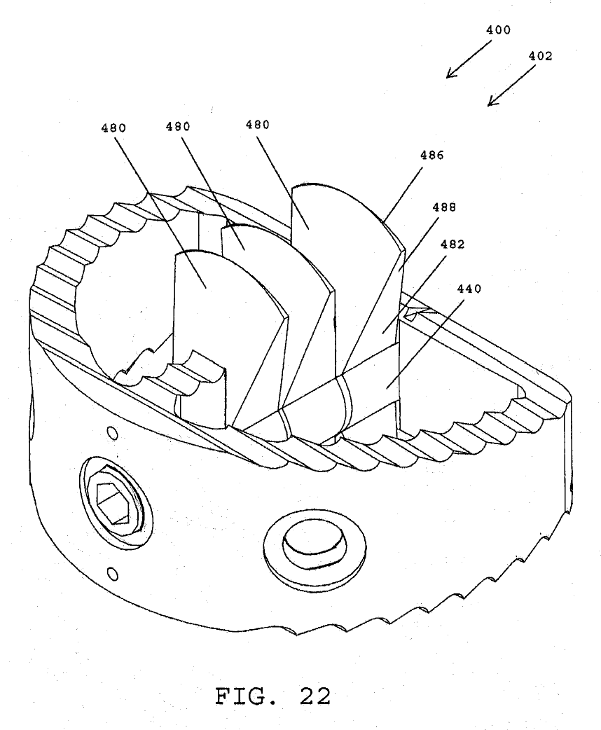

The configuration of the piercing portion 180 is not limited by the examples shown in FIGS. 1-31. It is contemplated that the piercing portion 180 can have any configuration capable of penetrating a vertebral body 10 and providing a secure connection. In particular, various configurations contemplated include a hook-shape as shown in FIG. 19, a fin shape as shown in FIGS. 22, 24, 29, an inverted triangle (preferably with the hypotenuse being the distal end portion), a “T” shape, an inverted “L” shape, or a disc-shape.

In one embodiment, as shown in FIG. 1, the piercing portion 180 includes a crook portion 184 intermediate the proximal portion 182 and the distal end portion 186. The crook portion 184 allows for the piercing portion 180 to have a longer configuration and be positionable within the central cavity 122 and, as a result, the piercing portion 180 extends further into the adjacent vertebral body 10. In a preferred embodiment, the crook portion 184 of the piercing portion 180 defines a radius of curvature of the piercing portion 180. Preferably, the radius of curvature is such that, when in the securing orientation, the distal end 186 of the piercing portion 180 is nearer the upper or lower surface 110, 112 of the implant body 102 than a portion of the piercing portion 180 intermediate the proximal portion 182 and the distal portion 186.

The crook portion 184 further aids in urging the implant device 100 toward the vertebral bodies 10 to produce a firm engagement between the vertebral bodies 10 and one or both of the upper and lower surfaces 110, 112. In particular, as the piercing portion 180 is rotated into the vertebral body 102 the distal end 186 extends away from the implant body 102 a distance determined by the length and radius of curvature of the proximal portion 180 and, after extending that distance, the distal end 186 of the piercing portion 180 rotates back toward the implant body 102. As the distal end 186 rotates back toward the implant body 102, the implant body 102 is urged toward the vertebral body 10, resulting in a more secure and flush engagement between the implant device 100 and the vertebral body 102.

According to one aspect of the invention, the implant device 100 includes a stop mechanism to secure the piercing portions 180 in the appropriate location within the adjacent vertebral body 10. Preferably, the stop mechanism is configured to either prevent over-rotation of the piercing portions 180 beyond the desired location or to prevent the piercing portion 180 from “backing-out” of the vertebral body 10 after the piercing portion 180 has been positioned in the desired location or from the resistance from the vertebral body 10 while the piercing portion 180 is being rotated into position, or both. Restricting the rotation of the piercing portions 180 after piercing the vertebral body 10 can further prevent micro-fissures within the vertebral body 10 and bone growth retardation.

In one embodiment, the stop mechanism is on the piercing portion 180. Back-out of the piercing portion 180 is prevented by the inclusion of a sharp projection extending backward obliquely off the forward facing piercing portion 180. In one embodiment, the distal end 186 of the piercing portion 180 is configured to include a locking mechanism 170 such as a hook, barb, or similar configuration which permits rotation of the piercing portion 180 in one direction but resists rotation in the opposite direction. In one preferred embodiment, the piercing portion 180 has a configuration of several overlapping triangles, the triangles overlapping along the length of the piercing portion and defining a number of barbs or hooks on either side of the triangle.

In an alternative embodiment, the stop mechanism causes mechanical interference to control rotation of the piercing portion 180. The stop mechanism can be configured to provide mechanical interference between the piercing portion 180 and the implant body 102, between the implant body 102 and the rotatable portion 140, or between the piercing portion 180 and the rotatable portion 140. In another preferred embodiment, the stop mechanism is configured to include a mechanical unlocking mechanism to allow for removal of the piercing portions 180 from the vertebral body 10 and for the removal of the implant device 100 from between the adjacent vertebrae. Examples of an unlocking mechanism include a button, lever, removable pin, a mechanical reversal or any other mechanically actuated mechanism suitable for such purpose.

In one embodiment, the stop mechanism includes an engagement surface of the central support portion 120. In particular, the engagement surface restricts the piercing portion 180 from over-rotation by abutting the piercing portion when the piercing portion 180 is rotated to the desired configuration.

In another embodiment, the stop mechanism includes a pin or screw member inserted into the vertebral body 10 to impede rotation or movement of the piercing portion 180. In one embodiment, the pin or screw member extends generally parallel to the upper and lower surfaces 110, 112 of the implant body. In another embodiment, the pin or screw member is accepted with a corresponding throughbore of the implant device 100. In an alternative embodiment, the pin or screw member is positioned adjacent the piercing portion 180, such as adjacent the crook portion 184, to impede movement of the piercing portion 180 within the implant body 102 and to impede rotation of the piercing portion 180 out from the implant body 102.

Other examples of stop mechanism configurations include a ratchet and pawl mechanism, rack and pinion, a mechanically actuated locking pin or a friction or snap fit connection between piercing portion 180 and rotatable portion 140, the piercing portion 180 and implant body 102, or the rotatable portion 140 and implant body 102.

According to yet another aspect, a plurality of gripping portions 118 may be formed on the upper and lower surfaces 110, 112 of the implant body 102 for engaging the adjacent vertebrae. As illustrated in FIGS. 1, 4, 5, the gripping portions 118 are defined in the upper and lower surfaces 110, 112 by a plurality of generally arcuate channels 119 extending generally perpendicular to the axis 142 of the implant body 102. In the illustrated form, the gripping portions 118 are uni-directional so that they assist insertion and resist explantation of the implant body 102. In alternative embodiments, for example, the gripping portions 118 include individual teeth. Further, in alternative embodiments, for example, the channels 119 extends in a direction which is not generally perpendicular to the axis 142, or the channels 119 extend in more than one direction.

Preferably, the gripping portions 118 are configured to be urged in engagement with the vertebral bodies by rotation of the piercing portion 180 into the vertebral bodies. As discussed above, as the piercing portion 180 rotatably penetrates the vertebral body 10. The implant body 102 and vertebral body 10 are urged toward each other into further engagement, thereby resisting explantation of the implant device 100 from between the adjacent vertebrae.

In one embodiment, the upper and lower surfaces 110, 112 of the implant body 102 are slanted with respect to each other so as to provide a generally wedge-shaped implant body 102 having a degree of lordosis. The degree of lordosis of the implant body 102 preferably corresponds to the natural lordosis of the lumbar spine. More specifically, the upper surface 110 has a line of lordosis extending through the upper leading edge 104 and the upper trailing edge 106 of the implant body 102, and lower surface 112 has a line of lordosis extending through the lower leading edge 104 and the lower trailing edge 106 of the implant body 102, such that the upper and lower surfaces 110, 112 are spaced apart a greater distance at the trailing edge 106 of the implant body 102 than at the leading edge 104 of the implant body 102, and the implant body has a height at the trailing edge 106 that is greater than a height at the leading edge 104.

Further, the line of lordosis of the upper surface 110 intersects the axis 142 of the implant body 102 at a first angle. Similarly, the line of lordosis of the lower surface 112 intersects the axis 142 of the implant body 102 at an second angle. The first and second angles may have any suitable size. Preferably, the first and second angles are sized to provide a degree of lordosis of the implant body 102 that best matches the natural lordosis of the spine. In one preferred form, the first angle is the same size as the second angle.

In another alternative embodiment, upper surface 110 and lower surface 112 are configured to be convex. The convex configuration of the upper surface 110 and the lower surface 112 may have any suitable convexity. The convexity is preferably selected to provide the best match to the natural concavity of the vertebral endplates.

Referring next to FIGS. 9-16, an alternative implant device 200 is shown. The following description will focus on the differences between the implant device 100 and the implant device 200, while a repeated description of the otherwise similar or identical features is generally omitted.

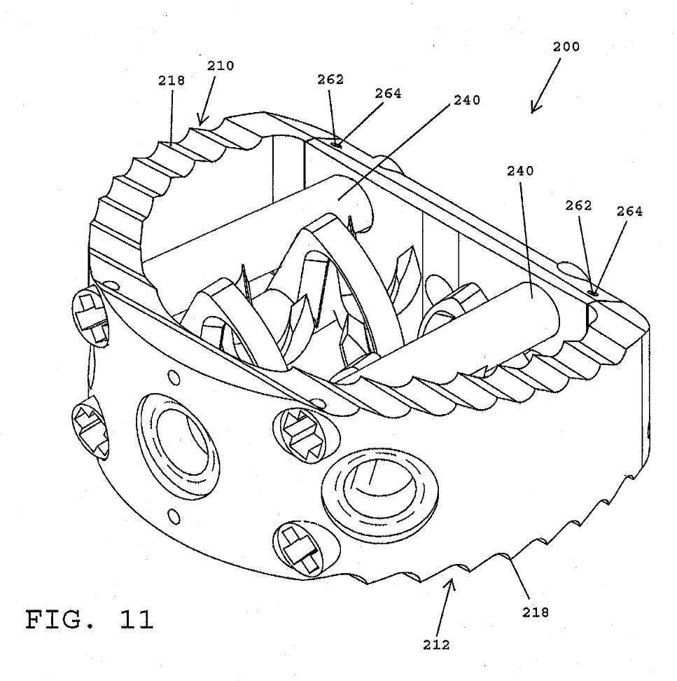

As in implant device 100, implant device 200 includes an implant body 202, a rotatable portion 240 and piercing portion 280. As shown in FIGS. 9, 10, piercing portions 280 extend in the opposite direction as the illustrated piercing portions 180. That is, piercing portion 280 include a distal end portion 286 which, when arranged in the securing orientation, extends toward the nearest lateral edge 208 (rather than extending toward the lateral edge 208 furthest from the piercing portion, as in implant device 100). As in implant device 100, and as shown in FIGS. 9, 11, implant device 200 preferably includes multiple rotatable portions 240 with at least one piercing portion 280 extending from each of the rotatable portions 240. By having the distal end portion 286 extend through the center portion of the vertebral body 10, which tends to be softer, and then extend to the outer portion of the vertebral body 10, which tends to be denser, the implant device 200 may be more firmly secured to the vertebral body 10.

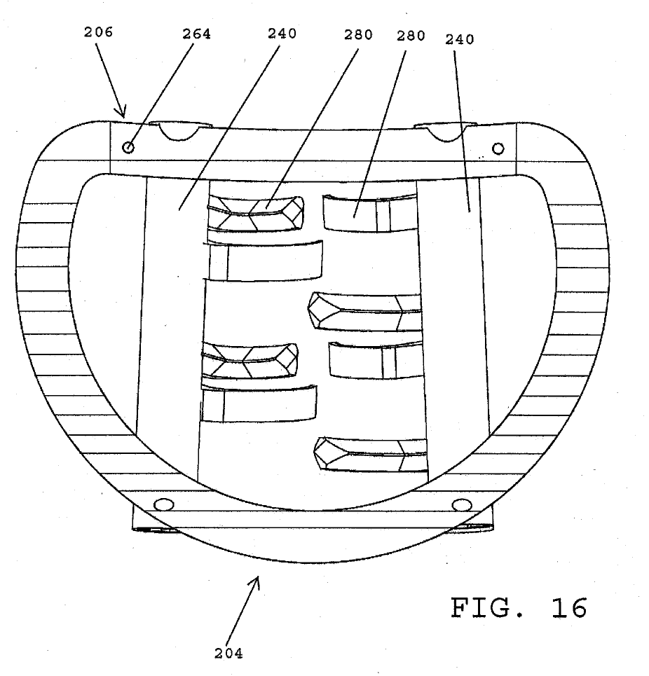

As shown in FIGS. 9, 11, 16, in order to accommodate the piercing portions 280 of implant device 200 within the central cavity 222, the central cavity 222 extends from lateral edge 208 to lateral edge 208, without a central wall or support portion therebetween. In the insertion orientation, the piercing portions 280, as illustrated in FIG. 16, extend across the central cavity 222 such that a central support or wall, as in implant device 100, would impede the piercing portions 280 from being positioned within the central cavity 222. However, it is contemplated that if the implant device 200 included piercing portions 280 which extended from only one of the upper and lower surfaces 210, 212, a central portion 220 could be included along the surface opposite the surface from which the piercing portions 208 extend in the securing orientation.

In addition, the implant body 202 can be configured with the throughbores 246, 248 of the leading and trailing edges 204, 206 positioned toward the lateral edges 208 to provide additional space in the central cavity 222 for the piercing portions 280 to be positioned while in the insertion orientation.

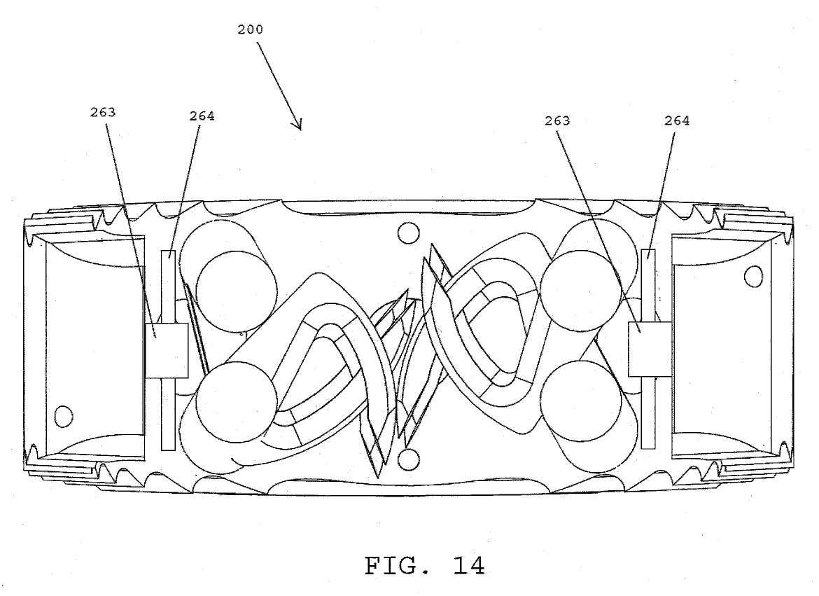

Further, as shown in FIG. 14, the leading edge 206 preferably includes a removable securing wall portion 260 that extends across the leading edge 206 and includes throughbore 248. The leading edge 206 further includes securing bosses 261 which correspond to securing slots 263 of the removable securing wall portion 260. A securing aperture 262 extends from the upper edge of the removable securing wall portion 260, through the securing wall portion 260 to the securing slot 263 and from the slot 263 through the removable securing wall portion 260 to the lower edge of the removable securing wall portion 260. A corresponding securing aperture 265 extends through the securing bosses 261 of the leading edge 206. The securing apertures 262, 265 are configured to accept a securing member 264, such as a pin, therein to secure the removable securing wall portion 260 to the leading edge 206 of the implant body 202.

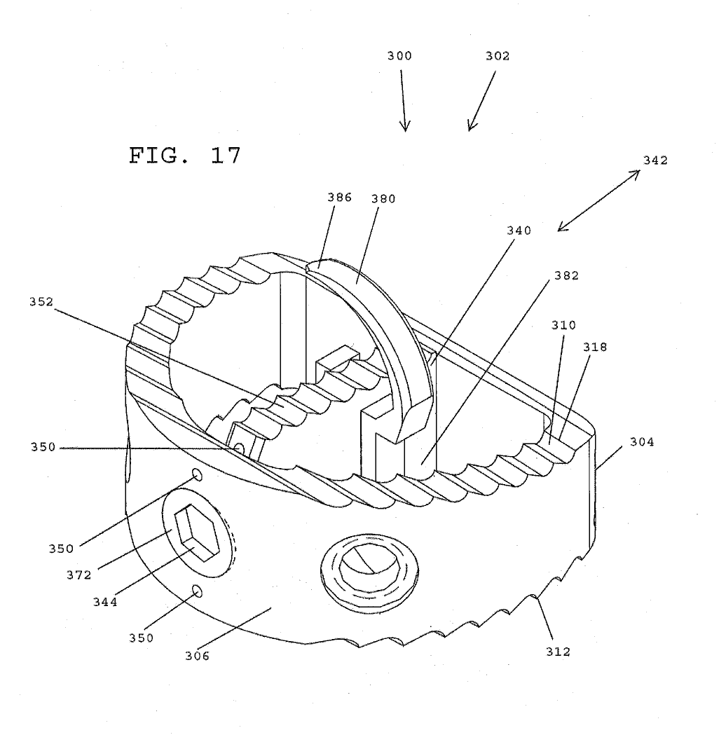

Referring next to FIGS. 17-21, an alternative implant device 300 is shown. The following description will focus on the differences between the implant device 100 and the implant device 300, with a repeated description of the otherwise similar or identical features generally omitted.

In this embodiment, as shown in FIGS. 17 and 19, the implant device 300 includes a rotatable portion 340 which, when in the securing orientation, also acts like the central support portion 120 of implant device 100. More particularly, the rotatable portion 340, when in the securing orientation, extends from the upper surface 310 to the lower surface 312 and from the leading edge 304 to the trailing edge 306. Preferably, the rotatable portion 340 includes gripping portions 318 corresponding to the gripping portions 318 of the implant body 302.

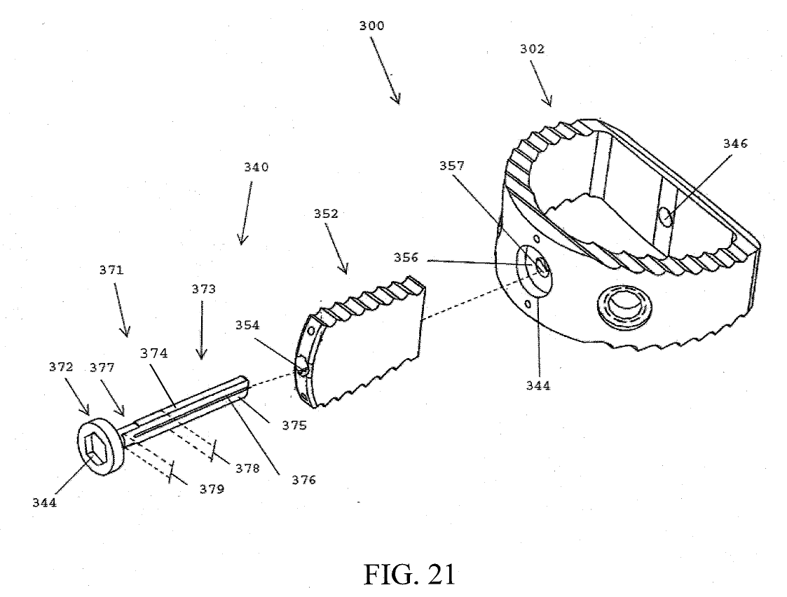

As shown in FIG. 21, the rotatable portion 340 includes a body portion 352 and an elongate securing portion 372. The body portion 352 includes a keyed throughbore 354 extending along the length of the body portion 352. The elongate securing portion 371 includes a head portion 372, a slotted portion 373 and a neck portion 377. The head portion 372 includes a tool engagement portion 344 therein. The slotted portion 373 includes an upper portion 374, a lower portion 375, a slot 376 extending along the lower and upper portions 374, 375 and a first height 378, and is configured to correspond to the keyed throughbore 354 of the body portion 352. The neck portion 377, which is intermediate the head portion 372 and slotted portion 373, includes a second height 379, the second height 379 being smaller than the first height 378.

The throughbore 346 of the trailing edge 306 includes a step 356 therein, the step 356 defining an annular throughbore having a step diameter 357. The annular throughbore of the step 356 is configured to accept the neck portion 377 therein and, in particular, to be larger than the second height 379 of the neck portion 377 and smaller than the head portion 372 and the first height 378 of the slotted portion 373.

The implant device 300 is assembled by inserting the slotted portion 373 of the elongate securing portion 371 through the throughbore 346 of the trailing edge 306. The upper and lower portions 374, 375 of the slotted portion 373 are urged together into the slot 376, effectively reducing the height 378 of the slotted portion 373 to less than the step diameter 357. The body portion 352 is positioned within the central cavity 322 of the implant body 302 to receive the slotted portion 373 within the keyed throughbore 354. The elongate securing portion 371 is shifted along the axis 342 until the slotted portion 373 is within the throughbore 348 of the leading edge 304, the neck portion 377 is disposed within the step 356 of the trailing edge 306, and the head 372 is disposed within the throughbore 346. The elongated securing portion 371 is thereby secured within the central cavity 322 both laterally, as the elongate securing portion 371 extends through throughbores 346, 348 thereby preventing lateral movement, and longitudinally, as the elongate securing portion 371 is positioned so that the head portion 372 and slotted portion 373 is on either side of the step 356, both the head portion 372 and slotted portion 373 sized larger than the diameter 357 of step 356 to prevent the elongate securing portion 371 from translating along the axis 342.

The keyed throughbore 354 and elongate securing portion 372 are configured to transmit torque applied by a tool 1000 engaging the tool engagement portion 344 to the body portion 352 of the rotatable portion 340. In particular, the upper and lower portions 374, 375 of the slotted portion 373 are configured to engage the keyed throughbore 354 and rotate the body portion 352 as rotational force is applied to the tool engagement portion 344.

A further embodiment of the piercing portion 380 is shown in FIG. 17, which includes a thinner and curved overall configuration. As with implant device 100, the piercing portion 380 can include various configurations based on the application and circumstances.

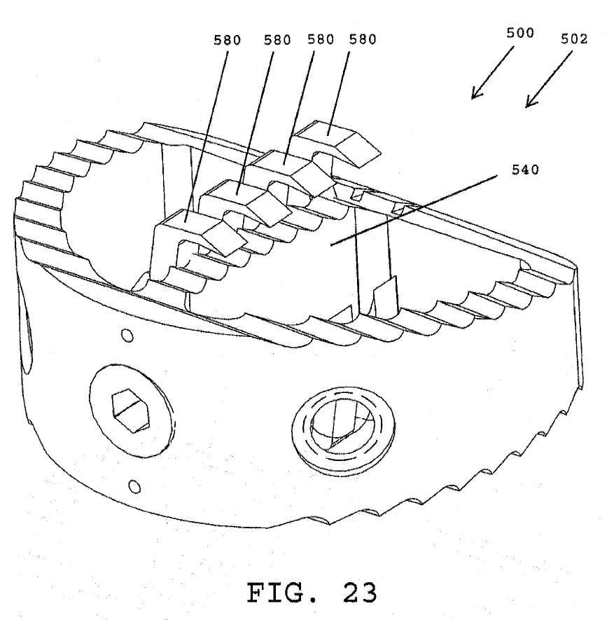

Referring next to FIG. 22, implant device 400, an alternative embodiment of implant device 300, is shown. In particular, implant device 400 includes piercing portions 480 having a wedge-shaped configuration. Referring to FIG. 23, implant device 500, an alternative embodiment of implant device 300 is shown. In particular, implant device 500 includes multiple piercing portions 580 extending from the upper and lower surfaces 510, 512.

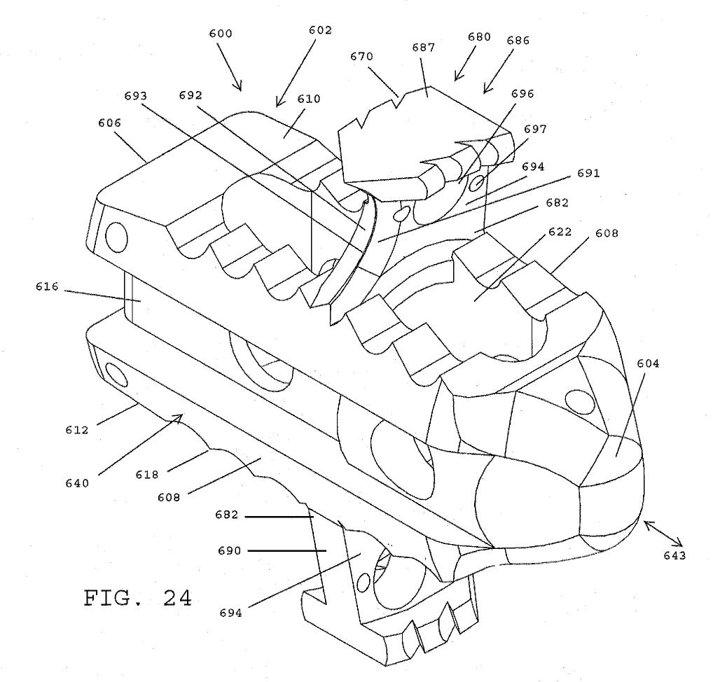

Referring next to FIGS. 24-28, an alternative implant device 600 is shown. The following description will focus on the differences between the implant device 100 and the implant device 600, with a repeated description of the otherwise similar or identical features generally omitted.

The implant device 600 includes an implant body 602 and piercing portions 680. The configuration of implant body 602 can include any implant device or artificial disc which is rotatable between adjacent vertebrae, and particularly the implants described in U.S. Patent Application Publication No. 2006/0129238 to Paltzer and U.S. Patent Application Publication No. 2007/0282441 to Stream et al., which are hereby incorporated in their entirety herein.

Generally, the implant body 602 includes a leading edge 604, a trailing edge 606 having a tool engagement portion 616, lateral edges 608, an upper surface 610, a lower surface 612, gripping portions 618, and a rotatable portion 640. The rotatable portion 640 of implant body 602 comprises the entire implant body 602, as the entire implant body 602 is rotatable between the adjacent vertebrae. In one embodiment, the implant body 602 does not include a central cavity as found in implant device 100. In the illustrated embodiment, the implant body defines a central cavity 622 positioned between the leading, trailing and lateral edges 604, 606 and 608 which preferably extends from the upper surface 610 to the lower surface 612. The implant body 602 also includes an axis 643 which is defined by the length 603 of the implant body 602.

The implant device 600 is configured to be inserted between adjacent vertebra with the lateral edges 608 in contact with the vertebral bodies, the upper and lower surfaces 610, 612 extending between the vertebral bodies and the piercing portions 680 extending from the upper and lower surfaces 610, 612. The piercing portions 680 are configured to extend a distance from the implant body 602 to provide adequate engagement with the vertebral bodies in the securing orientation while minimizing the space occupied by the piercing portions 680 when in the insertion orientation between the adjacent vertebrae. After the implant device 600 is positioned between the adjacent vertebrae, the implant body 602 is engaged by a tool at the tool engagement portion 616 and the entire implant device 600 is rotated along the axis 643 so that the piercing portions 680 penetrate the adjacent vertebrae and the upper and lower surfaces 610, 612 are in engagement with the adjacent vertebrae.

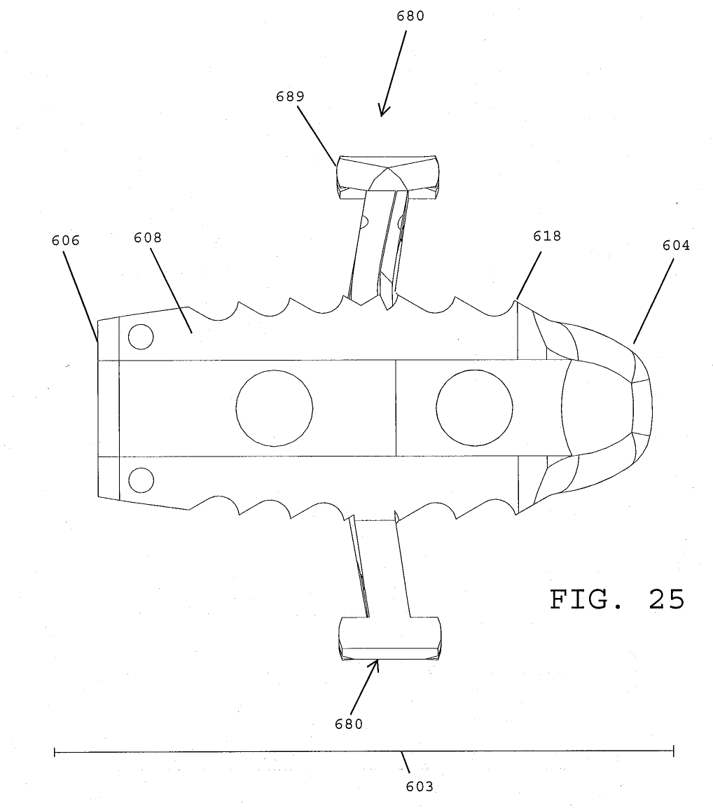

In one embodiment, as shown in FIGS. 24, 25, the leading edge 604 includes a tapered, contoured surface configured to ease insertion of the implant device 600 between adjacent vertebrae.

In a preferred embodiment, the implant body 602 includes rounded corners 609 along the intersection of the lateral edges 608 and the upper and lower surfaces 610, 612. Preferably, the rounded corners 609 are configured to assist in rotation of the implant body 602 between the adjacent vertebrae and reduce the risk of damage to the vertebral body 10 caused by the rotation of the implant device 600.

In another preferred embodiment, the lateral edges 208 include a convex surface 607 configured to ease insertion and rotation of the implant device 600 between the adjacent vertebrae.

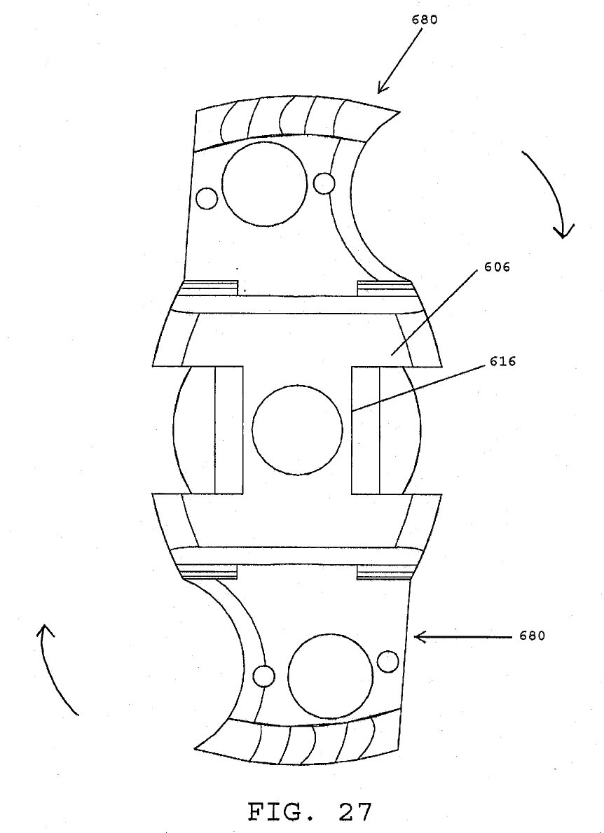

The implant device 600 further includes at least one piercing portion 680. Preferably, the implant device includes at least two piercing portions 680, such as shown in FIGS. 24, 27. Preferably, the implant device 600 includes at least one piercing portion 680 extending from the upper surface 610 and at least one piercing portion 680 extending from the lower surface 612.

The piercing portion 680 includes a proximal portion 682 and a distal end portion 686. The piercing portion 680 extends generally normal from the axis 643 of the implant body 602. In particular, as shown in FIG. 24, the proximal portion 682 of each piercing portions 680 is connected to one of the upper and lower surfaces 610, 612, and extends from one of the lateral edges 608, across the central cavity 622 and to the other lateral edge 608. In one embodiment, as shown in FIG. 24, the proximal portion 682 includes a rounded arc portion 683 extending across the central cavity 622.

The piercing portion 680 further includes a penetrating edge 691 extending from one of the lateral edges 608, a blunt edge 690 opposite the penetrating edge 691 extending from the other lateral edge 608, and a pair of opposing sidewalls 694 extending therebetween. The penetrating edge 691 is configured to ease penetration of the vertebral body 10 as the implant device 600 is rotated between the adjacent vertebrae. Preferably, the penetrating edge 691 has a tapered edge 692 as shown in FIG. 27. Further, it is preferable that the penetrating edge 691 is configured to ease penetration, such as by having a concave edge 692, as shown in FIG. 27. In a further preferable embodiment, the penetrating edge 691 is sharpened to facilitate insertion into the vertebral body 10.

As shown in FIGS. 24, 26, 27, in one embodiment the distal end 686 of the piercing portion 680 further includes a shelf 687 extending generally normal to the piercing portion 680 and generally parallel to the axis 643 of the implant device 600. In one embodiment, the shelf 687 extends outwardly toward the leading edge 604 of the implant body 602. In an alternative embodiment, the shelf 687 extends outwardly toward the trailing edge 606 of the implant body 600. Alternatively, the shelf 687 extends outwardly toward both the leading edge 604 and the trailing edge 606, as shown in FIG. 25. Finally, the shelf side ends 689 may be rounded, flat or tapered. In a preferable embodiment the shelf side ends 689 have a convex surface configuration, as shown in FIGS. 24, 25.

In one embodiment, as shown in FIG. 24, the shelf 687 includes a locking mechanism 670 in the form of cutout portions configured to resist migration of the implant device 600 from between adjacent vertebrae.

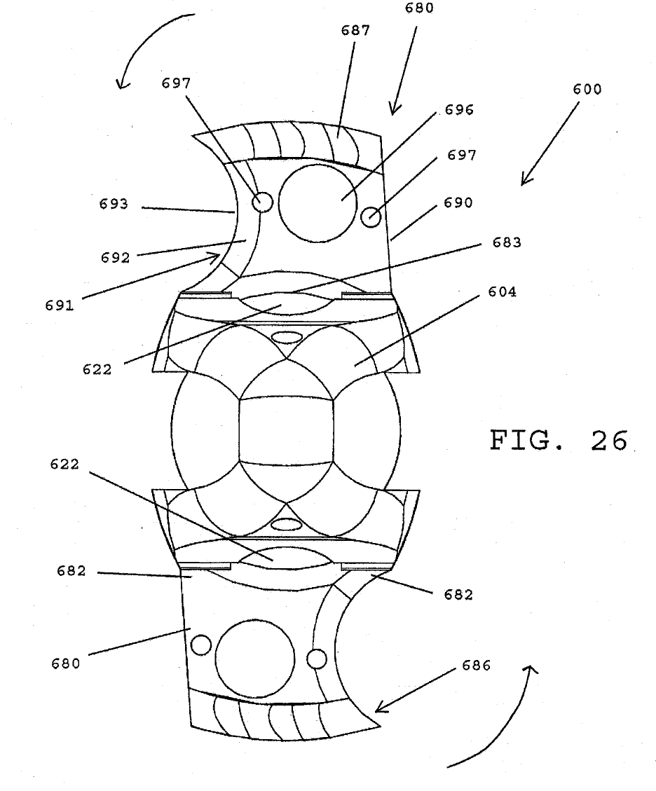

In an alternative embodiment, the piercing portions 680 are further secured within the vertebral bodies by a securing member (not shown). The securing member is configured to extend through the vertebral body 10 and a securing throughbore 696 of the piercing portion 680. Preferably, the securing member extends generally parallel to the axis 643. As shown in FIGS. 26, 27, the securing throughbore 696 extends from one sidewall 694 of the piercing portion 680 to the other sidewall 694, and is generally centrally located intermediate the blunt edge 690 and the piercing edge 691. In one embodiment, the piercing portion 680 further includes at least one small throughbore 697. The small throughbore 697 is preferably located adjacent the securing throughbore 696. In one embodiment, the small throughbore 697 and securing throughbore 696 define an axis that extends parallel to the shelf 687. In a preferred embodiment, the piercing portion 680 includes at least one small throughbore 697 between the blunt edge 690 and the securing throughbore 696 and at least one small throughbore 697 between the penetrating edge 691 and the securing throughbore 697, as shown in FIGS. 24, 26, 27. In one embodiment, for example, the small throughbores 697 are used to house additional, smaller securing member. In an alternative embodiment, the small throughbores 697 are configured to accept radiographic markers therein to assist in insertion of the securing member within the securing throughbore 696. In a further embodiment, the small throughbores 697 are configured to permit bone growth therethrough, and may be configured to accept bone growth promoting material therein.

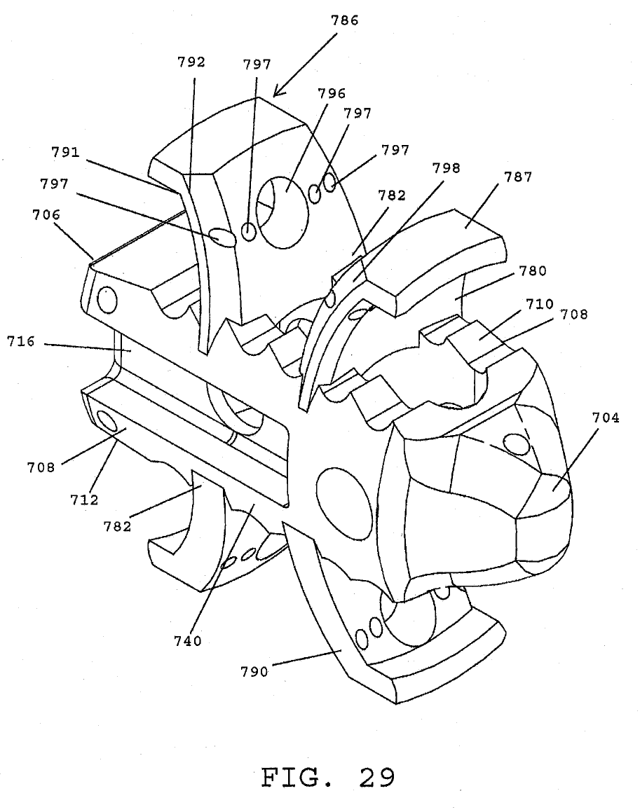

Referring next to FIGS. 29-31, an alternative implant device 700 is shown. The following description will focus on the differences between the implant device 600 and the implant device 700, with a repeated description of the otherwise similar or identical features generally omitted.

The implant device 700, as shown in FIG. 29, includes two piercing portions 780 extending from the upper surface 710 and two piercing portions 780 extending from the lower surface 712. The piercing portions 780 include a crook or curved portion 798 such that the piercing portions 780 extend away from the upper and lower surfaces 710, 712 and toward the closer of the leading and trailing edges 704, 706. It is contemplated that the piercing portions 780 include a curved portion 798 therein and extend in the same direction from the implant body 702, or, alternatively, that the piercing portions 780 would extend toward the central point of the implant body 702 along the axis 743. By having at least two piercing portions 780 extending in different directions, the implant device 700 is more secure between the adjacent vertebrae and is able to better resist explantation. The degree of curvature of the curved portion 798 is configured to provide a stable interface between the piercing portion 780 and the vertebral body 10 and to secure the implant device 700 between the adjacent vertebrae.

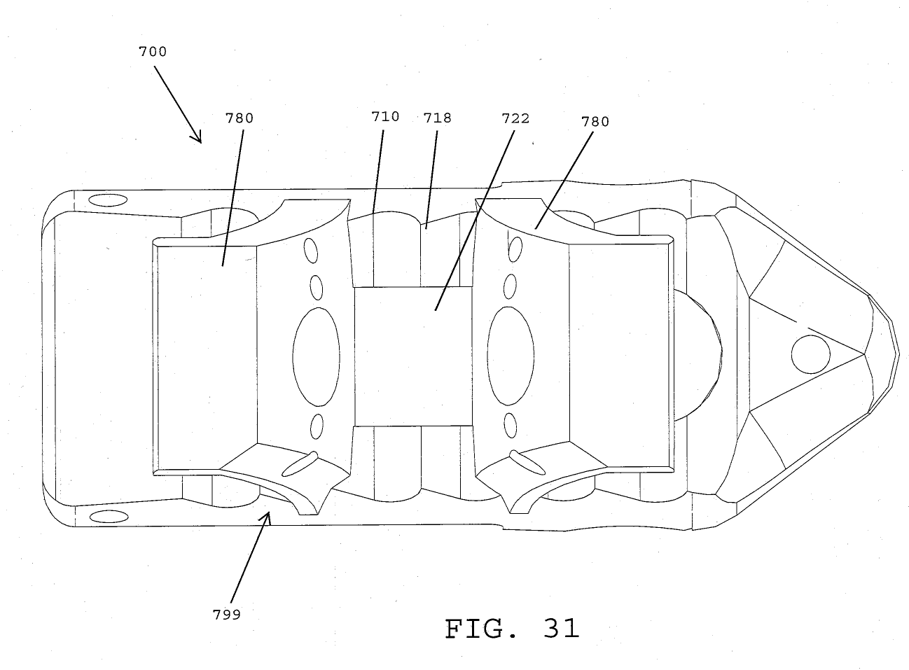

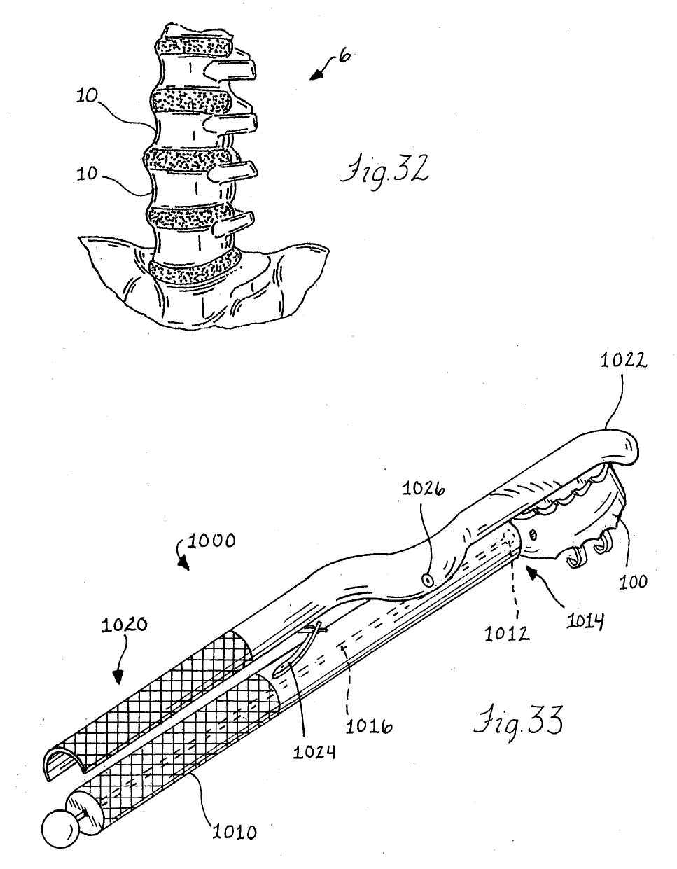

Additionally, the piercing portion 780 of the implant device 700, as shown in FIGS. 29, 31, includes a piercing edge 791 which is configured to include a convex configuration 799 to ease insertion into the vertebral body 10. In more detail, by configuring the piercing edge 791 so that the vertebral body 10 is first engaged by a small portion of the piercing edge 791, less torque is required to initially penetrate the vertebral body 10 than if the entire piercing edge 791 engages the vertebral body 10 at once. After the vertebral body 10 is initially penetrated, the convex configuration 799 of the piercing edge 791 provides for a gradual increase in the amount of the piercing edge 791 penetrating the vertebral body 10 until the entire piercing edge 791 is engaging the vertebral body 10.

The implant devices of the present invention may be fabricated from any suitable materials having desirable strength and biocompatibility. Suitable materials may include, for example, biocompatible metals and related alloys (such as titanium and stainless steel), shape memory metals (such as Nitinol), biocompatible polymers (including, for example, materials of the polyaryletherketone family such as PEEK (polyetheretherketone), PAEK (polyaryletherketone), PEK (polyetherketone), PEKK (polyetherketoneketone), PEKEKK (polyetherketoneetherketoneketone), PEEKK (polyetheretherketoneketone), and PAEEK (polyaryletheretherketone), filled materials (such as carbon or glass fiber-reinforced materials), bone substitute materials (such as hydroxyapatite and tricalcium phosphate), composite materials, and/or any combination of the above.

In one preferred form, the implant devices are formed of a PEEK-type material. In another from, the implant device may be formed, in whole or in part, or coated with a calcium phosphate ceramic bone substitute such as hydroxyapatite, tricalcium phosphate, and/or mixtures thereof. Particularly preferred hydroxyapatite and tricalcium phosphate compositions include those disclosed in, for example, U.S. Pat. No. 6,013,591, U.S. Pat. No. RE 39,196, and U.S. Patent Application Publication No. 2005/0031704, which are hereby incorporated in their entirety herein. Coating with the calcium phosphate ceramics can be achieved by any known method, including dip coating-sintering, immersion coating, electrophoretic deposition, hot isostatic pressing, solution deposition, ion-beam sputter coating and dynamic mixing, thermal spraying techniques such as plasma spraying, flame spraying and high-velocity oxy-fuel combustion spraying. In one preferred embodiment, hydroxyapetite coating is achieved by plasma spraying.

In yet another form, the implant device may be formed of a PEEK-type material and coated with such a bone substitute material. In yet another form, the implant device may be formed, in whole or in part, coated with, injected with, incorporate, and/or retain a bone growth stimulating composition such as the bioactive hydrogel matrix described, for example, in U.S. Pat. No. 6,231,881, U.S. Pat. No. 6,730,315, U.S. Pat. No. 6,315,994, U.S. Pat. No. 6,713,079, U.S. Pat. No. 6,261,587, U.S. Pat. No. 5,824,331, U.S. Pat. No. 6,068,974, U.S. Pat. No. 6,352,707, U.S. Pat. No. 6,270,977, U.S. Pat. No. 5,614,205, U.S. Pat. No. 6,790,455, U.S. Pat. No. 5,922,339, and U.S. Patent Application Publication No. 2005/0118230, which are hereby incorporated in their entirety herein.

Alternatively, the implant device of the invention may be formed of two distinct materials. In particular, the implant body may be formed of a first material, such as PEEK or carbon fiber PEEK, and the piercing portions may be made of a metal, such as Ti64. In one, the piercing portions of implant device 600, 700 are formed of a metal. Additionally, the part or the entire rotatable portion of the implant devices 100, 200, 300, 400, 500 may be formed of a material distinct from the material used to form the implant body.

The central cavity 122, 222, 322, 422, 522, 622, 722 provides a region for receiving bone growth material therein. In one embodiment, the implant device 100, 200, 300, 400, 500, 600, 700 is packed with bone growth filler prior to implantation. In a preferred embodiment, the implant device 100, 200, 300, 400, 500, 600, 700 can be implanted in the vertebral space and then packed with bone growth filler. Preferably, the bone growth material is inserted through the insertion tool engagement portion 416. In a alternative preferred embodiment, a bioresorbable sponge fixated to the implant device is used to secure the bone growth stimulating composition.

The bone void filler or graft material is preferably a combination of one or more various substances consisting of bone matrix, bone void filler, bone graft extender, biopolymers that stimulate bone growth, bone growth stimulating orthobiologic products, bioactive hydrogel matrix comprising a polypeptide and a long chain carbohydrate, and osteoinductive or osteoconductive materials, medicaments, stem or progenitor cells, and three-dimensional structural frameworks. In some embodiments, the bone matter may be a composition made from de-mineralized bone matrix.

In one embodiment, the bone growth stimulating composition comprises a bioactive hydrogel matrix comprising a polypeptide, such as gelatin, and a long chain carbohydrate, such as dextran, such as described in U.S. Pat. No. 6,231,881 to Usala et al. and U.S. Patent Application Publication No. 2005/0118230 to Hill et al., which are incorporated by reference in their entirety herein. In an alternative embodiment, this bone growth stimulating composition can be integrated with hydroxyapetite or other bone substitutes to provide sustained delivery of the bone growth stimulating compositions.

In one embodiment, the bone void fillers include a moldable putty optimized for implantation which provide significantly greater set time than most bone void fillers, such as one or both of TrioMatrix™ and FortrOss™. The increased set time allows the bone void filler, in the form of moldable putty optimized for implantation, to be extruded into the central cavity as the bone void filler remains “moldable” for a sufficient length of time. Furthermore, TrioMatrix™ and FortrOss™ have superior biological performance for inducing bone growth making them ideal as bone void fillers.

The bone void filler, such as TrioMatrix™, is preferably made from synthetically made hydroxyapatite, synthetically made gelatin carrier, demineralized bone matrix, and the patient's own blood products and/or bone marrow extract. In another form, the bone void filler, such as FortrOss™, is made from the mixing of synthetically made hydroxyapatite, synthetically made gelatin carrier, and the patient's own blood products and/or bone marrow extract.

The implant devices can readily be filled with such a moldable bone void filling putty. Moreover, biologic materials may be introduced to this admixture by the surgeon in the operating room, such as bone morphogenetic proteins (BMP) or bone growth stimulating compositions, to further induce bone growth. In yet another embodiment, bone chips from the patient can be added to the bone void filler.

Preferably, the bone void filler composition, such as FortrOss™, is made of synthetic and autograph materials to eliminate the risk of infection from bone donors and reduce the risk of rejection of the bone filler by the patient's immune system. Autograft materials are tissue that is transplanted from one portion of the patient's body to another. In the instant invention, bio-compatible autograft materials from the patient's own body in the form of blood products or bone chips with synthetic extenders of the autograft material are to be placed in the central cavity that encourage bone growth within and around the device.

Hydroxyapatite (HA) and tricalcium phosphate (TCP) can be used in the bone void filler for facilitating bone fusion. These compositions facilitate fusion by having the characteristic of being “bioactive” which indicates the ability to facilitate a cellular or tissue response, such as, induction of vasculogenesis, promotion of cellular attachment to a scaffold material, and promotion of tissue regeneration.

The previously described devices for securing an implant to bone will need to be implanted into the human body. The preferred embodiment of the apparatus 1000 for implanting a device for securing an implant to bone is shown in FIG. 33. The apparatus 1000 for implanting the device has a cannulated main shaft 1010 with a mechanism 1012 located on the distal end 1014 for attaching an implantable device. The distal end 1014 of the main shaft 1010 attaches to the device for securing an implant to bone to allow for minimally invasive surgery from various approaches through the patients body.

The main shaft 1010 has a rotatable rod 1016 located with the main shaft 1010 capable of longitudinal motion within the main shaft 1010. The rotatable rod 1016 allows the piercing portions to be locked into place. The longitudinal motion of the rod 1016 allows for disengagement of the apparatus 1000 from the device.

In addition, an arm 1020 with a counter-force plate 1022 located on the distal end 1014 for securing an implant on the main shaft 1010 is provided as shown in FIG. 33. The counter-force plate 1022 maintains attachment of the device during the insertion of the device into the patient. The plate 1022 is disengaged by compressing a spring 1024 located between the main shaft 1010 and the arm 1020. The main shaft 1010 and the arm 1020 are connected by a pin 1026 that allows the arm 1020 to hinge on the main shaft 1010.

The complete method for operating the device for securing an implant to bone begins with making a surgical incision, distracting the tissue in place, and removing the severely damage tissue. The device is then inserted and positioned in the patient. The rotatable portion is then rotated, along with the piercing portions, so that the piercing portions penetrate the adjacent vertebral bodies. The patient is then closed and the procedure is complete. The bone growth stimulating compounds, the other bone substitutes material, and the patients own body then heals the remaining wounds and causes the implanted device and adjacent bone to fuse into a solid structure to support the patient's body weight.

Those skilled in the art will recognize that a wide variety of modifications, alterations, and combinations can be made with respect to the above described embodiments without departing from the spirit and scope of the invention, and that such modifications, alterations, and combinations, are to be viewed as being within the scope of the invention.

What is claimed is:

1. A spinal implant for being implanted between adjacent vertebrae, the spinal implant comprising:
an implant body comprising a leading edge and a trailing edge;
a plurality of gripping portions extending from the implant body and configured to engage at least one of the adjacent vertebra;
a rotatable portion of the implant body extending generally from the leading edge to the trailing edge and defining an axis, the rotatable portion configured to be rotatable about the axis when the implant body is positioned between adjacent vertebrae; and
a piercing portion extending from the rotatable portion and configured to rotate about the axis and rotatably pierce one of the adjacent vertebrae.

2. The spinal implant of claim 1 comprising a plurality of piercing portions.

3. The spinal implant of claim 2 wherein each piercing portion extends in different predetermined directions from the implant body to facilitate a secure engagement of the implant to the bone.

4. The spinal implant of claim 2 wherein the implant body includes opposing surfaces for engaging adjacent vertebrae extending between the leading and trailing edges, at least two sets of at least two piercing portions extending from one of the opposing surfaces.

5. The spinal implant of claim 4 comprising distal ends of each of the piercing portions, the distal ends of each of the sets of at least two piercing portions facing toward each other.

6. The spinal implant of claim 2 wherein the implant body includes a pair of opposing surfaces for engaging adjacent vertebrae extending between the leading and trailing edges, at least one set of at least two piercing portions extending from each of the opposing surfaces.

7. The spinal implant of claim 6 wherein each of the piercing portions include distal ends facing generally in the same direction.

8. The spinal implant of claim 1 wherein the rotatable portion, leading edge, trailing edge and piercing portion are integral with one another.

9. The spinal implant of claim 1 wherein rotatable portion rotates independent of the implant body.

10. The spinal implant of claim 9 comprising a plurality of piercing portions and a plurality of rotatable portions.

11. The spinal implant of claim 9 comprising a ratcheting mechanism configured to permit rotation of the rotatable portion in one direction and restrict rotation in an opposite direction.

12. The spinal implant of claim 1 comprising a stop mechanism configured to resist rotation of the piercing portion away from the adjacent vertebra.

13. The spinal implant of claim 12 wherein the stop mechanism includes a barb at a distal end of the piercing portion.

14. The spinal implant of claim 12 wherein the piercing portion rotates independent of the implant body, and the stop mechanism includes a stop portion of the implant body configured to abut the piercing portion and restrict rotation thereof.

15. The spinal implant of claim 12 wherein the stop mechanism is a ratchet and pawl.

16. The spinal implant of claim 12 wherein the stop mechanism includes a recess extending through the piercing portion, the recess configured to receive a securing member therein.

17. The spinal implant of claim 16 wherein the securing member is an elongate member.

18. The spinal implant of claim 16 wherein the securing member is threaded.

19. The spinal implant of claim 12 wherein the stop mechanism includes a boss extending from the piercing portion, the boss configured to be engaged by a securing member extending through the vertebrae to resist migration of the piercing portion.

20. The spinal implant of claim 1 wherein the piercing portion includes a crook.

21. The spinal implant of claim 1 comprising a contoured surface of the implant body configured to provide a flush engagement with an adjacent vertebra.

22. The spinal implant of claim 21 wherein the plurality of gripping portions extend from the contoured surface.

23. The spinal implant of claim 21 comprising a pair of opposing contoured surfaces configured to provide a flush engagement with both adjacent vertebrae.

24. The spinal implant of claim 21 wherein the piercing portion extends from the contoured surface.

25. The spinal implant of claim 1 wherein the implant body includes a pair of body members each configured to engage one of the adjacent vertebrae and including a polyaxial interface therebetween.

26. The spinal implant of claim 1 wherein the implant body is configured to be positioned within an aperture in the annulus.

27. The spinal implant of claim 1 comprising a cavity extending through the implant body and configured to receive bone-growth promoting material therein.

28. The spinal implant of claim 1 wherein the implant body comprises a biocompatible material.

29. A spinal implant for being implanted between adjacent vertebrae, the spinal implant comprising:
an implant body comprising a leading edge and a trailing edge and defining a longitudinal axis therebetween;
a plurality of gripping portions extending from the implant body and configured to grip at least one of the adjacent vertebra; and
a piercing portion integral with the implant body and extending generally normal to the longitudinal axis, the implant body configured to rotate between adjacent vertebrae so that the piercing portion rotatably pierces one of the adjacent vertebrae.

30. The spinal implant of claim 29 wherein the piercing portion is a fin.
